SEQUENCE LISTING (45 sequences)

SEQ ID NO: 1 - RNA, 23 nt, Homo sapiens
agcuacauugucugcuggguuuc

SEQ ID NO: 2 - RNA, 21 nt, Homo sapiens
agcuacaucuggcuacugggu

SEQ ID NO: 3 - RNA, 22 nt, homo sapiens
aagcugccaguugaagaacugu

SEQ ID NO: 4 - RNA, 22 nt, Homo sapiens
uagcaccaucugaaaucgguua

SEQ ID NO: 5 - RNA, 20 nt, Homo sapiens
uaaggcacgcggugaaugcc

SEQ ID NO: 6 - RNA, 22 nt, Homo sapiens
ugaccugggacucggacagcug

SEQ ID NO: 7 - RNA, 17 nt, Homo sapiens
gggagaagggucggggc

SEQ ID NO: 8 - RNA, 22 nt, Homo sapiens
caucuggcauccgucacacaga

SEQ ID NO: 9 - RNA, 21 nt, Homo sapiens
ugacugccucacugaccacuu

SEQ ID NO: 10 - RNA, 22 nt, Homo sapiens
gccucucucggagucgcucgga

SEQ ID NO: 11 - RNA, 22 nt, Homo sapiens
ggggguccccggugcucggauc

SEQ ID NO: 12 - RNA, 22 nt, Homo sapiens
ugggucuuugcgggcgagauga

SEQ ID NO: 13 - RNA, 21 nt, Homo sapiens
uagcagcacagaaauauuggc

SEQ ID NO: 14 - RNA, 22 nt, Homo sapiens
auaccucaucuagaaugcugua

SEQ ID NO: 15 - RNA, 21 nt, Homo sapiens
aacauagaggaaauuccacgu

SEQ ID NO: 16 - RNA, 22 nt, Homo sapiens
aucacacaaaggcaacuuuugu

SEQ ID NO: 17 - RNA, 20 nt, Homo sapiens
guggguuggggcgggcucug

SEQ ID NO: 18 - RNA, 24 nt, Homo sapiens
ugggcgaggggugggcucucagag

SEQ ID NO: 19 - RNA, 22 nt, Homo sapiens
ugaaacauacacgggaaaccuc

SEQ ID NO: 20 - RNA, 20 nt, Homo sapiens
aaaagugauugcaguguuug

SEQ ID NO: 21 - RNA, 22 nt, Homo sapiens
aauucuguaaaggaagaagagg

SEQ ID NO: 22 - RNA, 19 nt, Homo sapiens
gagccaguuggacaggagc

SEQ ID NO: 23 - RNA, 22 nt, Homo sapiens
aucgggaaugucguguccgccc

SEQ ID NO: 24 - RNA, 22 nt, Homo sapiens
ugucuacuacuggagacacugg

SEQ ID NO: 25 - RNA, 22 nt, Homo sapiens
uggagugugacaaugguguuug

SEQ ID NO: 26 - RNA, 22 nt, Homo sapiens
cagugcaauguuaaaagggcau

SEQ ID NO: 27 - RNA, 20 nt, Homo sapiens
ucguuugccuuuuucugcuu

SEQ ID NO: 28 - RNA, 23 nt, Homo sapiens
gaacgcgcuucccuauagagggu

SEQ ID NO: 29 - RNA, 22 nt, Homo sapiens
cugggagaggguuguuuacucc

SEQ ID NO: 30 - RNA, 22 nt, Homo sapiens
aagacgggaggaaagaagggag

SEQ ID NO: 31 - RNA, 22 nt, Homo sapiens
agcuuccaugacuccugaugga

SEQ ID NO: 32 - RNA, 21 nt, Homo sapiens
gcagggacagcaaaggggugc

SEQ ID NO: 33 - RNA, 23 nt, Homo sapiens
ccugggcagcguguggcugaagg

SEQ ID NO: 34 - RNA, 21 nt, Homo sapiens
ggccagccaccaggagggcug

SEQ ID NO: 35 - RNA, 22 nt, Homo sapiens
cucuagagggaagcacuuucug

SEQ ID NO: 36 - RNA, 25 nt, Homo sapiens
ugccccaucugugcccuggguagga

SEQ ID NO: 37 - RNA, 23 nt, Homo sapiens
ugagugugugugugugagugugu

SEQ ID NO: 38 - RNA, 22 nt, Homo sapiens
ucacaagucaggcucuugggac

SEQ ID NO: 39 - RNA, 21 nt, Homo sapiens
ucgaggagcucacagucuagu

SEQ ID NO: 40 - RNA, 18 nt, Homo sapiens
ucgaggagcucacagucu

SEQ ID NO: 41 - RNA, 22 nt, Homo sapiens
cagugcaauguuaaaagggcau

SEQ ID NO: 42 - RNA, 21 nt, Homo sapiens
uuacaggcgugaaccaccgcg

SEQ ID NO: 43 - RNA, 23 nt, Homo sapiens
gaagugcuucgauuuuggggugu

SEQ ID NO: 44 - RNA, 20 nt, Homo sapiens
cgggcgugguggugggggug

SEQ ID NO: 45 - RNA, 24 nt, Homo sapiens
uggggagcugaggcucugggggug

(19) United States
(12) Patent Application Publication
(10) Pub. No.: US 2015/0005290 A1
(43) Pub. Date: Jan. 1, 2015

(54) COMPOSITIONS AND METHODS FOR DIAGNOSING AND TREATING SALT SENSITIVITY OF BLOOD PRESSURE

(71) Applicants: Robin A. Felder, Charlottesville, VA (US); Julia Carlson, Washington, DC (US); John J. Gildea, Waynesboro, VA (US)

(72) Inventors: Robin A. Felder, Charlottesville, VA (US); Julia Carlson, Washington, DC (US); John J. Gildea, Waynesboro, VA (US)

(73) Assignee: UNIVERSITY OF VIRGINIA PATENT FOUNDATION, Charlottesville, VA (US)

(21) Appl. No.: 14/316,183

(22) Filed: Jun. 26, 2014

Related U.S. Application Data
(60) Provisional application No. 61/839,525, filed on Jun. 26, 2013.

(51) Int. Cl. C12Q 1/68; A61K 31/554

(52) U.S. Cl. CPC C12Q 1/6883; A61K 31/554; C12Q 2600/178
USPC 514/211.07; 435/6.11

(57) ABSTRACT

To characterize the urinary exosome miRNome, microarrays were used to identify the miRNA spectrum present within urinary exosomes from ten individuals that were previously classified for their salt sensitivity status. The present application discloses distinct patterns of selected exosomal miRNA expression that were different between salt-sensitive (SS), salt-resistant (SR), and inverse salt-sensitive (ISS) individuals. These miRNAs can be useful as biomarkers either individually or as panels comprising multiple miRNAs. The present invention provides compositions and methods for identifying, diagnosing, monitoring, and treating subjects with salt sensitivity of blood pressure. The applications discloses panels of miRNAs useful for comparing profiles, and in some cases one or more of the miRNAs in a panel can be used. The miRNAs useful for distinguishing SS and SR or ISS and SR subjects. One or more of the 45 miRNAs can be used. Some of the miRNAs have not been previously reported to be circulating. See those miRNAs with asterisks in FIG. 1 and below. The present invention encompasses the use of one or more of these markers for identifying and diagnosing SR, SS, and ISS subjects.

### CROSS REFERENCE TO RELATED APPLICATIONS

This application claims priority under 35 U.S.C. §119(e) to U.S. Provisional Application Ser. No. 61/839,525 filed Jun. 26, 2013, the disclosure of which is incorporated by reference in its entirety herein.

### STATEMENT REGARDING FEDERALLY SPONSORED RESEARCH OR DEVELOPMENT

This invention was made with government support under Grant No. HL 074940, awarded by The National Institutes of Health. The government has certain rights in the invention.

### BACKGROUND

Salt sensitivity of blood pressure (BP) is associated with higher incidence of cardiovascular disease independent of hypertension [1]; however, it is difficult to diagnose. The most effective method is using an extensive two-week dietary protocol [2]. Finding a simpler method to correctly diagnose this condition is critical since it affects approximately 25% of the population [3], [4].

Exosomes are 50-90 nm membrane-derived vesicles found in bodily fluids including blood, saliva, and urine. They encapsulate proteins and mRNA as well as miRNA that may be exchanged as a signaling mechanism between cells [5]. Encapsulated mRNA and miRNA may be stable diagnostic biomarkers because exosomes protect nucleic acids from extracellular degradation [6], [7].

The relationship between salt intake and cardiovascular risk is not linear, but rather fits a J-shaped curve relationship. Thus, a low-salt diet may not be beneficial to everyone and may paradoxically increase blood pressure in some individuals. Accurate testing of salt sensitivity is not only laborious but also expensive, and results are not always accurate because of low patient compliance. Patients who have normal blood pressure but are salt-sensitive cannot be diagnosed in an office setting and there are no laboratory tests for salt sensitivity. The most reliable method to measure salt sensitivity is the blood pressure response to a change in dietary salt intake. There are currently no quick and cost effective methods to diagnose salt-sensitive (SS), salt-resistant (SR), and inverse salt-sensitive (ISS) individuals. Because of the difficulty in measuring the responses to sodium intake, surrogate markers are often used. Current surrogate markers of salt-sensitivity are not adequately sensitive or specific. The need for better surrogate markers for salt sensitivity other than PRA (plasma renin activity), ANP (atrial natriuretic peptide), BNP (brain natriuretic peptide), and endogenous ouabain still remains.

There is a long felt need in the art for compositions and methods useful for identifying, diagnosing, monitoring, and treating subjects with salt sensitivity of blood pressure. The present invention satisfies these needs.

### SUMMARY OF THE INVENTION

Pathophysiologic consequences related to the amount of dietary salt may be highly individualized but are difficult to predict. miRNAs have been characterized previously in total urine specimens and exosomes from body fluids other than urine, but have yet to be studied in urinary exosomes. Advances have been made in understanding the role of miRNAs in cancer pathogenesis, but less is known about their role in other chronic diseases. Studies have been reported associating certain miRNAs with hypertension [8] but miRNAs have not yet been directly linked to sodium metabolism. Potentially, miRNAs may be exchanged between tubule segments via exosomes to alter expression downstream in the nephron.

It is disclosed herein that urinary exosomes originating exclusively from the kidney provide organ-specific information about the contents of cellular cytoplasm and cellular physiology. A series of technical steps are provided herein for detecting kidney-derived miRNAs whose changes in levels are correlated with salt-sensitivity of blood pressure or to inverse salt-sensitivity based on a novel procedure disclosed herein to help eliminate readings due to other miRNAs that can be found in urine. That is, it is disclosed herein that urinary exosomes can be isolated and purified for the assay to avoid contamination from miRNAs originally from sources other than the kidney. In one embodiment, the present invention provides a method for detecting kidney-specific miRNAs in urinary exosomes prepared in the absence of a protease inhibitor comprising a method that is a modification of Gonzales (10) as follows:

1) centrifuging a urine sample that is less than about four hours old at about 17,000×g for 30 minutes to remove cells and debris;

2) obtaining a supernatant from step 1 and submitting the supernatant to ultracentrifugation at about 200,000×g for about 1 hour and optionally submitting a pellet obtained from said centrifugation to one or more rounds of ultracentrifugation;

3) resuspending the pellet of step 2 into phosphate buffered saline and isolating total RNA from said resuspended pellet; and

4) submitting said RNA to microarray analysis and detecting and quantifying miRNA in said sample.

To characterize the urinary exosome miRNome, microarrays were used to identify the miRNA expression spectrum present within urinary exosomes from ten individuals that were previously classified for their salt sensitivity status. Potential biomarkers were sought based on the three salt sensitivity categories described herein. The present application discloses distinct patterns of selected exosomal miRNA expression that were different between salt-sensitive (SS), salt-resistant (SR), and inverse salt-sensitive (ISS) individuals. These miRNAs can be useful as biomarkers either individually or as panels comprising multiple miRNAs.

Detecting and measuring miRNAs from purified urinary exosomes is a novel feature over the current art and is useful for diagnosing and comparison purposes for salt sensitivity or inverse salt-sensitivity as well as the presence of or a predisposition to cardiovascular disease. Provided herein is a series of miRNAs useful for these procedures. The present invention provides a technological advance over the expensive and time consuming procedure commonly used where changes in blood pressure are measured in response to changes in dietary sodium intake or to the assays measuring surrogate markers.

In one embodiment, the present invention provides compositions and methods for identifying, diagnosing, monitoring, and treating subjects with salt-sensitivity of blood pressure or inverse salt-sensitivity. The methods can be used to differentiate SR, ISS, and SS subjects and to characterize their miRNA profile. The present application discloses biomarkers as well as novel combinations of biomarkers useful for identifying, diagnosing, monitoring, and treating subjects with salt sensitivity of blood pressure or with inverse salt-sensitivity. The present application further discloses novel miRNA profiles for SR, SS, and ISS subjects, based on changes in levels of one or more miRNAs in SS and ISS subjects compared to SR subjects.

In one embodiment, once urinary exosomes have been isolated and purified, a sample can be processed to reverse transcribe a miRNA in a serum sample, amplifying the miRNA, measuring the level of the miRNA, wherein the miRNA has a sequence selected from the group consisting of SEQ ID NO:1-45, and detecting whether the level of the miRNA is higher or reduced, as compared to a level from a salt-resistant subject or to a control or standard value, thereby characterizing whether a subject is salt-sensitive or inverse salt-sensitive.

Forty-five of the most important miRNAs of the invention have been assigned sequence identification numbers (SEQ ID NO) as provided below in Table I:

TABLE I
45 miRNAs

| Reporter | Target Seq | SEQ. ID. |
|---|---|---|
| Name | (5′ to 3′) | NO. |
| hsa-miR-221-3p | AGCUACAUUGUCU | 1 |
| | GCUGGGUUUC | |
| hsa-miR-222-3p | AGCUACAUCUGGC | 2 |
| | UACUGGGU | |
| hsa-miR-22-3p | AAGCUGCCAGUUG | 3 |
| | AAGAACUGU | |
| hsa-miR-29a-3p | UAGCACCAUCUGA | 4 |
| | AAUCGGUUA | |
| hsa-miR-124-3p | UAAGGCACGCGGU | 5 |
| | GAAUGCC | |
| hsa-miR-3661 | UGACCUGGGACUC | 6 |
| | GGACAGCUG | |
| hsa-miR-4516 | GGGAGAAGGGUCG | 7 |
| | GGGC | |
| hsa-miR-3126-3p | CAUCUGGCAUCCG | 8 |
| | UCACACAGA | |
| hsa-miR-5002-3p | UGACUGCCUCACU | 9 |
| | GACCACUU | |
| hsa-miR-3183 | GCCUCUCUCGGAG | 10 |
| | UCGCUCGGA | |
| hsa-miR-615-5p | GGGGGUCCCCGGU | 11 |
| | GCUCGGAUC | |
| hsa-miR-193a-5p | UGGGUCUUUGCGG | 12 |
| | GCGAGAUGA | |
| hsa-miR-195-5p | UAGCAGCACAGAA | 13 |
| | AUAUUGGC | |
| hsa-miR-4777-3p | AUACCUCAUCUAG | 14 |
| | AAUGCUGUA | |
| hsa-miR-376c | AACAUAGAGGAAA | 15 |
| | UUCCACGU | |
| hsa-miR-377-3p | AUCACACAAAGGC | 16 |
| | AACUUUUGU | |
| hsa-miR-3940-5p | GUGGGUUGGGGCG | 17 |
| | GGCUCUG | |
| hsa-miR-4649-5p | UGGGCGAGGGGUG | 18 |
| | GGCUCUCAGAG | |
| hsa-miR-494 | UGAAACAUACACG | 19 |
| | GGAAACCUC | |
| hsa-miR-548ah-5p | AAAAGUGAUUGCA | 20 |
| | GUGUUUG | |
| hsa-miR-4778-5p | AAUUCUGUAAAGG | 21 |
| | AAGAAGAGG | |
| hsa-miR-575 | GAGCCAGUUGGAC | 22 |
| | AGGAGC | |
| hsa-miR-425-3p | AUCGGGAAUGUCG | 23 |
| | UGUCCGCCC | |
| hsa-miR-934 | UGUCUACUACUGG | 24 |
| | AGACACUGG | |
| hsa-miR-22-5p | UGGAGUGUGACAA | 25 |
| | UGGUGUUUG | |
| hsa-miR-30a-3p | CAGUGCAAUGUUA | 26 |
| | AAAGGGCAU | |
| hsa-miR-1282 | UCGUUUGCCUUUU | 27 |
| | UCUGCUU | |
| hsa-miR-523-3p | GAACGCGCUUCCC | 28 |
| | UAUAGAGGGU | |
| hsa-miR-30c-1-3p | CUGGGAGAGGGUU | 29 |
| | GUUUACUCC | |
| hsa-miR-483-5p | AAGACGGGAGGAA | 30 |
| | AGAAGGGAG | |
| hsa-miR-2115-5p | AGCUUCCAUGACU | 31 |
| | CCUGAUGGA | |
| hsa-miR-211-3p | GCAGGGACAGCAA | 32 |
| | AGGGGUGC | |
| hsa-miR-3187-5p | CCUGGGCAGCGUG | 33 |
| | UGGCUGAAGG | |
| hsa-miR-3194-5p | GGCCAGCCACCAG | 34 |
| | GAGGGCUG | |
| hsa-miR-518d-5p | CUCUAGAGGGAAG | 35 |
| | CACUUUCUG | |
| hsa-miR-3189-5p | UGCCCCAUCUGUG | 36 |
| | CCCUGGGUAGGA | |
| hsa-miR-574-5p | UGAGUGUGUGUGU | 37 |
| | GUGAGUGUGU | |
| hsa-miR-125b-2-3p | UCACAAGUCAGGC | 38 |
| | UCUUGGGAC | |
| hsa-miR-151a-5p | UCGAGGAGCUCAC | 39 |
| | AGUCUAGU | |
| hsa-miR-151b | UCGAGGAGCUCAC | 40 |
| | AGUCU | |
| hsa-miR-130a-3p | CAGUGCAAUGUUA | 41 |
| | AAAGGGCAU | |
| hsa-miR-5095 | UUACAGGCGUGAA | 42 |
| | CCACCGCG | |
| hsa-miR-373-3p | GAAGUGCUUCGAU | 43 |
| | UUUGGGGUGU | |
| hsa-miR-1268b | CGGGCGUGGUGGU | 44 |
| | GGGGGUG | |
| hsa-miR-939 | UGGGGAGCUGAGG | 45 |
| | CUCUGGGGGUG | |

The assay and markers disclosed herein are also useful for predicting risk of cardiovascular disease. Furthermore, it is disclosed herein using the compositions and methods of the invention that 24 miRNAs (see FIG. 1 and Table II) that were not previously described as secreted from cells or even found in body fluids can be found in urinary exosomes. The present invention therefore includes an assay for identifying miRNAs using urinary exosomes that are specific to kidney function.

These 24 miRNAs specific to the kidney exosome are useful as a panel or profile of the kidney and comprises miRNAs having the following sequences: 6, 7, 8, 9, 10, 14, 16, 17, 18, 20, 21, 24, 27, 29, 31, 32, 33, 34, 35, 36, 40, 42, and 44.

In addition to discovering that urinary exosomes comprise miRNAs not known to be secreted miRNAs, the present application discloses that, depending on whether a subject is salt-resistant (SR), salt-sensitive (SS) or inverse salt-sensitive (ISS), one or more miRNAs disclosed herein isolated from urinary exosomes can be detected and measured to distinguish SR, SS and ISS subjects from one another.

In one embodiment, the present invention provides a panel of 45 miRNAs (SEQ ID NOs:1-45) useful for distinguishing SS, ISS, and SR subjects. In one aspect, one or more of the 45 miRNA markers can be detected and measured at a time. In one aspect, all 45 can be screened as a panel. In one aspect, depending on the phenotype or diagnosis of interest being assayed, any combination of 2 or more of the 45 of the miRNAs of the invention can be detected and measured based on the specific combination that may be needed to determine the phenotype. In one aspect, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of the miRNAs are detected and measured. In one aspect, the present invention encompasses a method for diagnosing a disease or disorder associated with a change in the level or expression of one or more miRNAs associated with a change in sensitivity to salt, with the proviso that when more than one is being detected and measured that they can be tested individually or as a group. Furthermore, when a diagnosis or prediction is being made for a specific group, one or more of the miRNAs shown herein to decrease or increase for that group can be used singly or in combination, based on the results disclosed herein. One of ordinary skill in the art will also understand that when only one or two miRNAs are being detected and measured, for example, if using has-miR-4516 (SEQ ID NO:7), has-miR-3183 (SEQ ID NO:10), has-miR-3940-5p (SEQ ID NO:17) and has-miR-4649-5p (SEQ ID NO:18), which are described herein to vary in expression levels in both SS and ISS subjects, an additional miRNA may need to be measured to differentiate SS subjects from ISS subjects.

In one embodiment, one to four miRNAs of the 45 miRNAs disclosed herein are useful for distinguishing between SR and ISS or SS.

In one embodiment, one or more of 24 unique kidney exosome miRNAs are useful for the practice of the invention for distinguishing the difference between SR and SS subjects. One or more of these can be used as biomarkers. The present invention encompasses the use of one or more of these markers for identifying and diagnosing SR and SS subjects.

In one embodiment, the present invention provides compositions and methods for determining whether a subject is salt-sensitive. It is an indication that a subject is salt-sensitive (SS) if one or more of an miRNA having a sequence of SEQ ID NOs:1-11 is higher in the subject relative to the levels in a SR subject or to a control or standard level. SEQ ID NOs:1-11 represent miRNAs-hsa-miR-221-3p, hsa-miR-222-3p, hsa-miR-22-3p, hsa-miR-29a-3p, hsa-miR-124-3p, hsa-miR-3661, hsa-miR-4516, hsa-miR-3126-3p, hsa-miR-5002-3p, hsa-miR-3183, and hsa-miR-615-5p, respectively. In one aspect, 2, 3, 4, 5, 6, 7, 8, 9, 10, or 11 of the miRNAs are detected and measured. One of ordinary skill in the art will appreciate that if all of the miRNAs detected and measured are not higher that the subject may not be SS.

In another embodiment, the present invention provides compositions and methods for determining whether a subject is salt-sensitive (SS). It is an indication that a subject is salt-sensitive if one of more of an miRNA having a sequence of SEQ ID NOs:12-24 is lower in the subject compared to the levels in a salt-resistant (SR) subject or to a control or standard level. SEQ ID NOs:12-24 represent miRNAs-hsa-miR-193a-5p, hsa-miR-195-5p, hsa-miR-4777-3p, hsa-miR-376c, hsa-miR-377-3p, hsa-miR-3940-5p, hsa-miR-4649-5p, hsa-miR-494, hsa-miR-548ah-5p, hsa-miR-4778-5p, hsa-miR-575, hsa-miR-425-3p, and hsa-miR-934, respectively. In one aspect, 1, 2, 3, 4, 5, 6, 7, 8, 9, 10, 11, 12, or 13 of the miRNAs are detected and measured. One of ordinary skill in the art will appreciate that if all of the miRNAs detected and measured are not lower, then the subject may not be SS.

One of ordinary skill in the art will appreciate that the two different salt-sensitive assays, or use of the two different groups of miRNAs, can also be combined. In one embodiment, a subject is salt-sensitive (SS) if: 1) one or more of an miRNA having a sequence of SEQ ID NOs:1-11 is higher in the subject compared to the levels in a salt-resistant (SR) subject or to a control or standard level; and 2) one of more of an miRNA having SEQ ID NOs:12-24 is lower in the subject compared to the levels in a salt-resistant (SR) subject or to a control or standard level.

In one embodiment, one or more of 21 of the 45 total miRNAs are useful for distinguishing the difference between SR and ISS subjects.

In one embodiment, the present invention provides compositions for determining if a subject is inverse sensitive to salt (ISS). It is an indication that a subject is inverse salt-sensitive if one or more of an miRNA having a sequence of SEQ ID NOs:10 and 25-37 is higher in the subject relative to the levels in a SR subject or to a control or standard level. SEQ ID NOs:10 and 25-37 represent miRNAs hsa-miR-3183, hsa-miR-22-5p, hsa-miR-30a-3p, hsa-miR-1282, hsa-miR-523-3p, hsa-miR-30c-1-3p, hsa-miR-483-5p, hsa-miR-2115-5p, hsa-miR-211-3p, hsa-miR-3187-5p, hsa-miR-3194-5p, hsa-miR-518d-5p, hsa-miR-3189-5p, and hsa-miR-574-5p, respectively. In one aspect, each miRNA is detected and is higher. In one aspect, two or more of the miRNAs are detected and measured. In one aspect, 1, 2, 3, 4, 5, 6, 7, 8, 9, 10, 11, 12, 13, 14, 15, 16, 17, or 18 of the miRNAs are detected and measured. One of ordinary skill in the art will appreciate that if all of the miRNAs detected and measured are not higher, then the subject may not be ISS.

In another embodiment, the present invention provides compositions for determining if a subject is inverse-sensitive to salt (ISS). It is an indication that a subject is inverse salt-sensitive if one or more of an miRNA having a sequence of SEQ ID NOs:7, 17, 18, and 38-45 is lower in the subject compared to the levels in a SR subject or to control or standard levels. SEQ ID NOs:7, 17, 18, and 38-45 represent miRNAs hsa-miR-4516, hsa-miR-3940-5p, hsa-miR-4649-5p, hsa-miR-125b-2-3p, hsa-miR-151a-5p, hsa-miR-151b, hsa-miR-130a-3p, hsa-miR-5095, hsa-miR-373-3p, hsa-miR-1268b, and hsa-miR-939, respectively. In one aspect, each miRNA is detected and is lower. In one aspect, 1, 2, 3, 4, 5, 6, 7, 8, 9, 10, or 11 of the miRNAs are detected and measured. One of ordinary skill in the art will appreciate that if all of the miRNAs detected and measured are not lower, then the subject may not be ISS.

The two ISS assays (based on the two panels of miRNAs that change) can also be combined and performed at the same time. It is also an indication that a subject is inverse-sensitive if one or more of an miRNA having a sequence of SEQ ID NOs:10 and 25-37 is higher in the subject relative to the levels in a SR subject or to a control or standard level and if one or more of an miRNA having a sequence of SEQ ID NOs:7, 17, 18, and 38-45 is lower in the subject compared to the levels in a SR subject or to control or standard levels.

Has-miR-4516 (SEQ ID NO:7), has-miR-3183 (SEQ ID NO:10), has-miR-3940-5p (SEQ ID NO:17) and has-miR-4649-5p (SEQ ID NO:18) stand out from the other miRNAs disclosed herein because each has been found to vary in expression levels in both SS and ISS subjects. For example, if multiple markers are to be used, but not all markers are being used, one of ordinary skill in the art will appreciate that SEQ ID NOs:17 and 18 should not be used alone because a decrease in each relative to the levels in an SR individual or to control or standard levels could mean that the subject is either SS or ISS. Without the use of additional markers SS and ISS subjects might not be distinguishable. It should also be noted that SEQ ID NO:10 increases in both SS and ISS subject relative to SR subjects, so it should not be used alone or even in combination with just SEQ ID NOs:17 and 18.

In one embodiment, one miRNA is useful for distinguishing three categories of salt sensitivity (SR, SS, and ISS) from one another. In one aspect, the miRNA is has-miR-4516 (SEQ ID NO:7). When has-miR-4516 is found at increased levels in a subject relative to its levels in a control subject or in a subject who is salt-resistant (SR), it is an indication that the subject is salt-sensitive (SS). If, however, has-miR-4516 is found to be decreased in a subject relative to has-miR-4516 levels in a control subject or in a subject who is salt-resistant (SR), it is an indication that the subject is inverse salt-sensitive (ISS).

In one embodiment, has-miR-3183 (SEQ ID NO:10) is higher in SS and ISS subjects compared to SR subjects. To distinguish SS from ISS, if has-miR-3183 is measured at least one more miRNA must be measured.

In one embodiment, has-miR-3940-5p (SEQ ID NO:17) and has-miR-4649-5p (SEQ ID NO:18) are lower in both SS and ISS subjects relative to the levels in SR subjects. To distinguish SS and ISS subjects from one another if either of these two miRNAs are detected and measured, at least one more miRNA must be measured.

In one embodiment, SEQ ID NOs:10, 17, and 18 can be used in combination to distinguish SS and ISS subjects from SR subjects.

Neither 4516, 3183, 3940-5p, nor 4649-5p (SEQ ID NO:7, 10, 17, and 18, respectively) has ever been reported to be a secreted miRNA. Applicants have identified herein miRNAs (from urinary exosomes) whose levels are related to salt-sensitivity or inverse salt-sensitivity, relative to salt-resistant individuals. Furthermore, the assay only uses urinary exosomes, not miRNA from other sources.

The present invention further provides for determining whether a subject is SR by measuring at least one of the 45 miRNAs of Table I herein and if no change is detected then the subject is SR.

The present invention provides a quick, accurate, and reliable assay compared to the dietary salt intake and blood pressure method or to the use of surrogate markers to determine salt-sensitivity. The present invention further provides for the simultaneous testing of SS and ISS and for determining whether a subject is SR.

Because some of the miRNAs are at higher levels in one type of subject compared to another, the set of miRNAs found at higher levels can be used as a group of one or more miRNAs to practice the methods of the invention. Because some of the miRNAs were at lower levels in one type of subject compared to another, the set of miRNAs found at lower levels can be used as a group of one or more miRNAs to practice methods of the invention. One of ordinary skill in the art will appreciate that one of more miRNAs from either high or low groups can also be combined for use as a panel of biomarkers.

In one embodiment, a diagnosis of the invention is substantiated by further tests. For example, the diagnosis can be substantiated by measuring blood pressure response to a change in dietary salt intake or by measuring at least one surrogate marker selected from the group consisting of plasma renin activity (PRA), atrial natriuretic peptide (ANP), and brain natriuretic peptide (BNP), or a combination of BP response and surrogate markers.

One of ordinary skill in the art will appreciate that the useful miRNAs disclosed herein can be used in conjunction with other miRNAs, including any that have not yet been found to correlate with salt-sensitivity of blood pressure or with inverse salt-sensitivity.

In one embodiment, each of the miRNAs having a sequence of SEQ ID NOs:1-45 is identified and measured.

One of ordinary skill in the art will appreciate that the useful miRNAs disclosed herein as identified in the urinary exosome can be detected and measured in conjunction with plasma-specific biomarkers to exclude plasma contamination. In one aspect, the plasma-specific biomarkers are exosomal biomarkers. In one aspect, the plasma-specific exosomal biomarkers may be used to exclude plasma contamination from occult glomerular disease. In one aspect, the biomarkers are miRNAs.

The miRNA results obtained from test subjects can be used to formulate treatment regimens and to determine useful drugs for treating a subject in need thereof. A treatment regimen can be designed and implemented based on whether salt-sensitivity or insensitivity is diagnosed. Once treatment is initiated, subjects can be assessed again (monitored) for the miRNAs or for response to the treatment and/or to intake of salt using, for example, blood pressure measurements, and the treatment can be adjusted accordingly. In one aspect, the information obtained can be reported to the subject or the subject's physician. Useful drugs of the invention include, but are not limited to, Diuretics, Combination Diuretics, ACE Inhibitors, ARBs (angiotensin II receptor blockers), Calcium Channel Blockers (CCBs), Central Agonists, Peripheral-Acting Adrenergic Blockers, and Direct Renin Inhibitors. In one aspect, at least two drugs may be administered to the subject. Two or more drugs may be indicated for use based on the miRNA profile obtained for SEQ ID NOs:1-45. In one aspect, at least three drugs are used. In another aspect, at least 4 drugs are prescribed.

In another embodiment, a diagnosis of SS or ISS based on an miRNA profile or expression levels of miRNA can be substantiated as such by, for example, administering salt or a drug treatment and determining the response. The response can be measured, for example, by determining blood pressure, salt levels in the subject, etc.

In one embodiment, the results of the assay can be used to determine if a patient is SS or ISS and this information can be used for, example, prescribing a dietary or fitness regimen or a combination of the above.

The present invention further provides kits for use in identifying and diagnosing subjects with one of the salt issues disclosed herein. In one aspect, a kit can include reagents for detecting one or more of the 45 miRNAs disclosed herein that can be used to distinguish SS, ISS, and SR subjects and an instructional material for the use thereof.

Various aspects and embodiments of the invention are described in further detail below.

### BRIEF DESCRIPTION OF THE DRAWINGS

FIG. 1. Differential miRNA expression in urine exosomes. miRNA expression patterns of 11 pooled urines were analyzed using miRBase version 18 microarray. In the heat maps on the left, each column corresponds to the relative expression profile of a pooled urine sample from an individual previously indexed for their salt sensitivity status. Replicates of specimens per individual are included in the heat map. Student's t-test was used to identify mature miRNAs that were significantly different between subjects (P<0.05). Panel A shows the 24 miRNAs that were different between the Salt-Resistant (SR) and Salt-Sensitive (SS) individuals. The 11 miRNAs above the horizontal line showed increased expression in the SS vs. the SR group, and the 13 miRNAs below that line showed decreased expression compared to the SR control. Panel B shows the 25 miRNAs (an additional 21) that were different between the Salt-Resistant (SR) and Inverse Salt-Sensitive (ISS) individuals. To the right of the reporter name, asterisks indicate 13 miRNAs in Panel A and 15 (11 additional) miRNAs in Panel B that have not previously been reported to be circulating. The bar graph on the right displays the absolute RFU of each specific miRNA probe. The top axis displays the values for the low expressers, and a different scale is used for the high expressing miRNAs. This is displayed on the bottom axis, for the bars to the right of the dotted line. The microarray hierarchical cluster analysis is depicted along the left edge of the figure by grouping genes that had the most similar expression patterns.

### DETAILED DESCRIPTION

### Abbreviations and Acronyms

ANP—atrial natriuretic peptide

BNP—brain natriuretic peptide

BP—blood pressure

ISS—inverse salt-sensitive

MAP—mean arterial pressure

miRNA—microRNA

PRA—plasma renin activity

SR—salt-resistant

SS—salt-sensitive

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the invention or a prodrug of a compound of the invention to a subject in need of treatment.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

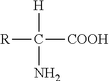

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, sputum, mucus, phlegm, tissues, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. One of skill in the art will understand the type of sample needed.

A “biomarker” or “marker” is a specific biochemical in the body which has a particular molecular feature that makes it useful for measuring the progress of disease or the effects of treatment, or for measuring a process of interest.

As used herein, the term “carrier molecule” refers to any molecule that is chemically conjugated to the antigen of interest that enables an immune response resulting in antibodies specific to the native antigen.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

A “computer-readable medium” is an information storage medium that can be accessed by a computer using a commercially available or custom-made interface. Exemplary compute-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

By “a control or standard level” is meant a level of a particular miRNA that has been determined to be a normal or standard level or amount so that it can be used as a standard for comparison of the level of the miRNA when measured in a test subject.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

“miRNAs” are RNA molecules of about, for example, 22 nucleotides or less in length. These molecules are post-transcriptional regulators that bind to complementary sequences on target mRNAs. Although miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules.

The term “negative response” to salt loading means a decrease in MAP upon sodium intake, particularly high sodium intake.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, D.C., p. 574).

The term “salt sensitivity of blood pressure” is a quantitative trait in which an increase in sodium (Na+) load leads to an increase in blood pressure (BP)

A “sample,” as used herein, refers preferably to a biological sample from a subject for which an assay or other use is needed, including, but not limited to, normal tissue samples, diseased tissue samples, sputum, mucus, phlegm, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. A sample can also be any other source of material obtained from a subject which contains cells, tissues, or fluid of interest. A sample can also be obtained from cell or tissue culture.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

A “variant”, as described herein, refers to a segment of DNA that differs from the reference DNA. A “marker” or a “polymorphic marker”, as defined herein, is a variant. Alleles that differ from the reference are referred to as “variant” alleles.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Embodiments

Total miRNA (miRNome) in urinary exosomes has not been previously evaluated as a disease bio-marker, particularly for salt sensitivity, but experiments described herein disclose novel and unexpected results.

Generally, salt sensitivity of blood pressure is a quantitative trait in which an increase in sodium (Na+) load leads to an increase in blood pressure (BP). In one aspect, salt sensitivity is about a ≧7 mm Hg increase in mean arterial pressure (MAP) during a randomized transition between high and low Na+ diet.

In one embodiment, the present invention encompasses diagnosing salt sensitivity of blood pressure or paradoxical responses to salt intake. In one aspect, the results of the diagnostic tests can be used for guidance in developing a treatment strategy for a subject who is diagnosed with salt sensitivity of blood pressure or paradoxical responses to salt intake.

In one embodiment, the invention provides for a method of determining the existence of salt sensitivity of blood pressure or a paradoxical response to salt intake comprising analyzing a sample from at test subject for the presence or absence of one or more of the miRNAs disclosed herein, wherein the one or more miRNAs is associated with salt sensitivity of blood pressure or a paradoxical response to salt intake. In one aspect, nucleic acid from the sample is analyzed. In one aspect, the nucleic acid is sequenced. In one aspect, the sample is a biological sample obtained from the subject. In one aspect, the sample is isolated from urine excreted by the subject.

The present invention is based in part on an assay utilizing miRNAs found in urinary exosomes, something not previously knows. In fact, the assay of the invention is based solely on miRNAs isolated from urinary exosomes, not miRNAs isolated from other tissues or fluids. In one aspect, miRNA expression levels of the biomarkers/SEQ ID NOs: listed are quantified with a biological assay. Assay can include RT-PCR, quantitative real-time-PCR, northern analysis, microarray analysis, and cDNA-mediated annealing, selection, extension, and ligation assay. One of ordinary skill in the art can determine which assay to use. A statistically validated threshold can be used to help differentiate SS, ISS, and SR subjects. The data obtained can also be used to assess risk for cardiovascular disease and for planning and initiating treatment regimens. Depending on how great the change in levels is, severity of SS or ISS can be determined.

Data obtained can also be used to predict the response of a subject to a treatment as well as to design a treatment for the subject. Treatment can include the use of drugs, change in diet, change in salt intake, or other methods to cause the miRNAs to return to a normal level.

With the addition of analogs such as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tm) of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′ end, or in the middle), the Tm could be increased considerably.

Treatment

The detection of the miRNAs described herein can be further used to help establish a treatment regimen and for monitoring treatment. One of ordinary skill in the art will appreciate that a variety of drugs, or combinations of drugs, can be used based in the age, health, and other characteristics of the subject. A diet comprised of salt concentrations or amounts that do not exceed each subject's personal salt index can also be established to control sodium intake.

For example, a subject found to be sensitive can be treated with one or more diuretics, mineralocorticoid receptor antagonists, beta-blockers, alpha-blockers, ACE inhibitors, angiotensin II receptor blockers, calcium channel blockers, central agonists, peripheral-acting adrenergic blockers, direct vasodilators, or direct renin inhibitors. Use of these drugs can also be coupled with a diet to limit sodium intake.

The present invention further encompasses the use of drugs such as fenofibrate, which has been recently shown to lower blood pressure in salt-sensitive but not salt resistant hypertension (Gilbert et al., 2013, J. Hypertension).

The types of drugs, and some specific drugs, useful in the present invention include, but are not limited to:

Diuretics—Some examples are:

Aldactone (spironolactone)

Dyrenium (triamterene)

Esidrix, Hydrodiuril, and Microzide (hydrochlorothiazide or HCTZ)

Hygroton and Thalitone (chlorthalidone)

Lasix (furosemide)

Lozol (indapamide)

Midamor (amiloride hydrochloride)

Mykrox and Zaroxolyn (metolazone)

Combination diuretics are also encompassed by the methods of the invention and include, but are not limited to:

Aldactazide (spironolactone and hydrochlorothiazide)

Dyazide and Maxzide (hydrochlorothiazide and triamterene)

Moduretic (amiloride hydrochloride and hydrochlorothiazide)

Beta-Blockers—Examples of beta-blockers include, but are not limited to:

Blocadren (timolol)

Cartrol (carteolol hydrochloride)

Coreg (carvedilol)

Corgard (nadolol)

Inderal (propranolol)

Kerlone (betaxolol)

Levatol (penbutolol sulfate)

Lopressor and Toprol XL (metoprolol)

Sectral (acebutolol)

Tenormin (atenolol)

Visken (pindolol)

Zebeta (bisoprolol fumarate)

Normodyne and Trandate (labetolol)

Alpha-Blockers—Examples of alpha-blockers, include, but are not limited to:

Cardura (doxazosin)

Hytrin (terazosin)

Minipress (prazosin)

ACE Inhibitors—Angiotensin-converting enzyme inhibitors (ACE) are high blood pressure medications that prevent production of angiotensin II. Examples of ACE inhibitors include, but are not limited to:

Accupril (quinapril)

Altace (ramipril)

Capoten (captopril)

Mavik (trandolapril)

Lotensin (benazepril)

Monopril (fosinopril)

Prinivil and Zestril (lisinopril)

Univasc (moexipril)

Vasotec (enalapril)

ARBs (angiotensin II receptor blockers)—Examples of ARBs, include, but are not limited to:

Atacand (candesartan)

Avapro (irbesartan)

Benicar (olmesartan)

Cozaar (losartan)

Diovan (valsartan)

Micardis (telmisartan)

Teveten (eprosartan)

Calcium Channel Blockers (CCBs)—Examples of CCBs include, but are not limited to:

Adalat and Procardia (nifedipine)

Calan, Covera, Isoptin, Verelan, and others (verapamil)

Cardene (nicardipine)

Cardizem, Cartia, Dilacor, and Tiazac (diltiazem)

DynaCirc (isradipine)

Norvasc (amlodipine)

Plendil (felodipine)

Sular (nisoldipine)

Central Agonists—These medications target receptors. Examples of central agonists include, but are not limited to:

Aldomet (methyldopa)

Catapres (clonidine)

Tenex (guanfacine)

Wytensin (guanabenz)

Peripheral-Acting Adrenergic Blockers—Examples of peripheral-acting adrenergic blockers include, but are not limited to:

Hylorel (guanadrel)

Ismelin (guanethidine)

Serpasil (reserpine)

Direct Vasodilators—Examples of direct vasodilators:

Apresoline (hydralazine)

Loniten (minoxidil)

Direct Renin Inhibitors—Direct renin inhibitors, ACE inhibitors, and ARBs all target the same process that narrows blood vessels. However, each type of medication blocks a different part of the process. Direct renin inhibitors block the enzyme renin from triggering a process that helps regulate blood pressure. Tekturna (aliskiren) is a direct renin inhibitor. Tekturna can be used alone or in combination with a diuretic or other medicines for high blood pressure.

The dosage of the active compound(s) being administered will depend on the condition being treated, the particular compound, and other clinical factors such as age, sex, weight, and health of the subject being treated, the route of administration of the compound(s), and the type of composition being administered (tablet, gel cap, capsule, solution, suspension, inhaler, aerosol, elixir, lozenge, injection, patch, ointment, cream, etc.). It is to be understood that the present invention has application for both human and veterinary use.

For example, in one embodiment relating to oral administration to humans, a dosage of between approximately 0.1 and 300 mg/kg/day, or between approximately 0.5 and 50 mg/kg/day, or between approximately 1 and 10 mg/kg/day, is generally sufficient, but will vary depending on such things as the disorder being treated, the length of treatment, the age, sex, weight, and/or health of the subject, etc. The drugs can be administered in formulations that contain all drugs being used, or the drugs can be administered separately. In some cases, it is anticipated that multiple doses/times of administration will be required or useful. The present invention further provides for varying the length of time of treatment.

Methods for Predicting Response to Therapeutic Agents

As is known in the art, individuals can have differential responses to a particular therapy (e.g., a therapeutic agent or therapeutic method). Another aspect of the invention relates to methods of selecting individuals suitable for a particular treatment modality, based on the likelihood of developing particular complications or side effects of the particular treatment. It is well known that most therapeutic agents can lead to certain unwanted complications or side effects. Likewise, certain therapeutic procedures or operations may have complications associated with them. Complications or side effects of these particular treatments or associated with specific therapeutic agents can, just as diseases do, have a genetic component. It is therefore contemplated that selection of the appropriate treatment or therapeutic agent can in part be performed by determining the urinary exosomal miRNA profile of an individual, and using that profile of the individual to decide on a suitable therapeutic procedure or on a suitable therapeutic agent to treat the particular disease. It is therefore contemplated that the markers of the invention can be used in this manner. In particular, the markers of the invention can be used to determine whether administration of a particular therapeutic agent or treatment modality or method is suitable for the individual, based on estimating the likelihood that the individual will develop symptoms associated with, for example, salt sensitivity of blood pressure or other conditions associated with paradoxical responses to salt intake as a consequence of being administered the particular therapeutic agent or treatment modality or method. Indiscriminate use of such therapeutic agents or treatment modalities may lead to unnecessary and needless adverse complications. The invention includes kits for assessing exosomal miRNA profiles as disclosed herein.

### Examples

### Materials and methods

Research Participants

Ten Caucasian subjects previously evaluated for their salt sensitivity status [2] were asked to participate in this study one to five years after their initial classification. The three phenotypes identified were: salt-sensitive (SS, N=3) who showed a ≧7 mm increase in mean arterial pressure (MAP) following an increase in sodium intake; salt-resistant (SR, N=4) who had <7 mm change in MAP following any change in sodium intake; and inverse salt-sensitive (ISS, N=3) whose MAP increased ≧7 mm following a decrease in sodium intake [3], [9]. Random urine samples were pooled from three to four independent collections from each subject. Two independent miRNA analyses were performed by microarray.

Exosome Purification

The ultracentrifugation protocol to isolate exosomes from urine samples was followed according to Gonzales et al. [10] with the following modifications: 1) protease inhibitors were not used because miRNA was the target and 2) the first centrifugation step to remove whole cells and debris was performed for 30 minutes rather than 10 minutes to ensure maximum purity. Urine specimens were processed as quickly as possible after voiding (<4 hours). Previous studies had demonstrated that miRNA is stable for 24 hours in urine at room temperature [11]. After exosome pellets were resuspended in Phosphate Buffered Saline, total RNA was isolated using a miRNeasy kit (Qiagen). Samples were stored individually at −80° C. until microarray analysis was performed.

miRNome Analysis

Three to four collections per individual were pooled in order to reach the 5 μg miRNA minimum required for enrichment and pre-amplification by a commercial firm (LC Sciences, Houston, Tex.). Individual miRNA expression was analyzed using microarray (miRBase Human: Version 18). To test for analytical variability of the miRNA microarray chips, each pooled specimen from 5 individuals was split into two aliquots and each aliquot was analyzed twice. The remaining 5 individuals were analyzed once in this first run. To get a preliminary understanding of biological variability, we collected and pooled specimens from the same 5 individuals (3 months later) and repeated the procedure of splitting the pools into two aliquots and analyzing them in duplicate. The other 5 individuals were examined once in this second run.

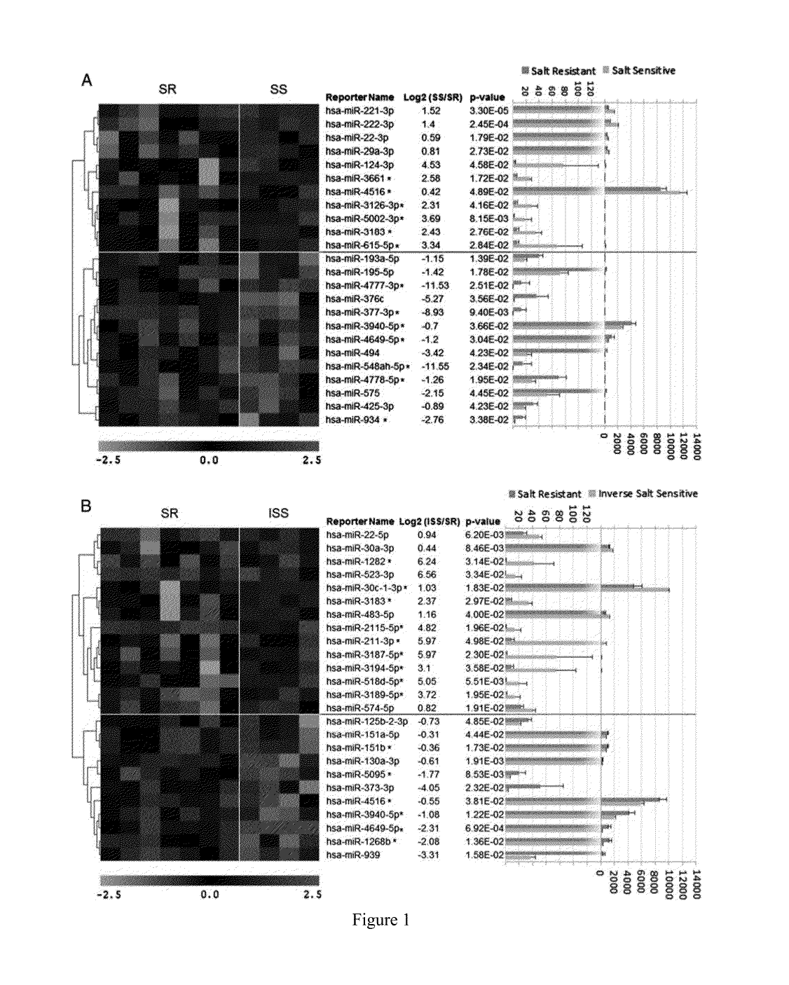

We compared the data from the individuals split into the three salt-sensitive phenotypes. The graphs are displayed as SS individuals compared to SR individuals (FIG. 1A), and ISS individuals compared to SR individuals (FIG. 1B).

Results

Out of the 1898 unique miRNA probes examined by the chips for miRBase Version 18, a total of 194 were above background in all ten subjects. These 194 miRNAs replicated within individual aliquots and between the analytical runs performed 3 months apart. Of the 194 miRNAs detected, 49 (when total between the two sets) were significantly different between the SS and SR individuals or between the ISS and SR subjects. The differences in urinary exosomal miRNAs between those groups replicated between pooled sample aliquots and also replicated between the analytical runs performed 3 months apart.

Four of the miRNAs shared significant expression differences between SR and ISS or SS. The only single miRNA that could differentiate the 3 categories of salt sensitivity was hsa-miR-4516, which was higher in SS and lower in ISS, compared to the SR group. One was higher in SS and ISS vs. SR (hsa-miR-3183) and 2 were lower in SS and ISS vs. SR (hsa-miR-3940-5p, hsa-miR-4649-5p).

We searched the literature for each of the 49 miRNAs that were significantly different between SS or ISS vs. SR individuals, and found that only 21 were previously cited as extracellular secreted miRNAs in serum or other bodily fluids (see the University of Catania website “miRandola”). The 24 miRNAs that have not been described as secreted from cells or found in body fluids are highlighted by asterisks in the 2 panels of FIG. 1. Currently, these miRNAs can be considered unique to urinary exosomes and strongly indicate that serum exosomal miRNAs do not pass into the urine in appreciable quantities. Six of the mature miRNAs that showed significant differences using microarrays were verified using quantitative PCR. Parallel significant difference were found using real-time PCR, matching the microarray analyses.

Our data demonstrate that there are significant differences in 49 miRNA concentrations in urinary exosomes, presumably derived from kidney tubule cells. The relative concentration of each miRNA is shown by pseudocolor representation on the heat maps at the left in each figure. There were dramatic differences between the various miRNA concentrations so the bar graph was made and scaled to accommodate these differences.

The following Table (Table II) provides the name, SEQ ID NO, sequence, and accession number of the miRNAs described above. Each asterisk indicates an miRNA that has not previously found to be secreted/circulating (see also FIG. 1).

TABLE II

| | | SEQ. | |
|---|---|---|---|
| Reporter | Target Seq | I.D. | |
| Name | (5′ to 3′) | NO. | Accession No. |
| hsa-miR-221-3p | AGCUACAUUGUCU | 1 | MIMAT0000278 |
| | GCUGGGUUUC | | |
| hsa-miR-222-3p | AGCUACAUCUGGC | 2 | MIMAT0000279 |
| | UACUGGGU | | |
| hsa-miR-22-3p | AAGCUGCCAGUUG | 3 | MIMAT0000077 |
| | AAGAACUGU | | |
| hsa-miR-29a-3p | UAGCACCAUCUGA | 4 | MIMAT0000086 |
| | AAUCGGUUA | | |
| hsa-miR-124-3p | UAAGGCACGCGGU | 5 | MIMAT0000422 |
| | GAAUGCC | | |
| hsa-miR-3661* | UGACCUGGGACUC | 6 | MIMAT0018082 |
| | GGACAGCUG | | |
| hsa-miR-4516* | GGGAGAAGGGUCG | 7 | MIMAT0019053 |
| | GGGC | | |
| hsa-miR-3126-3p* | CAUCUGGCAUCCG | 8 | MIMAT0015377 |
| | UCACACAGA | | |
| hsa-miR-5002-3p* | UGACUGCCUCACU | 9 | MIMAT0021024 |
| | GACCACUU | | |
| hsa-miR-3183* | GCCUCUCUCGGAG | 10 | MIMAT0015063 |
| | UCGCUCGGA | | |
| hsa-miR-615-5p* | GGGGGUCCCCGGU | 11 | MIMAT0004804 |
| | GCUCGGAUC | | |
| hsa-miR-193a-5p | UGGGUCUUUGCGG | 12 | MIMAT0004614 |
| | GCGAGAUGA | | |
| hsa-miR-195-5p | UAGCAGCACAGAA | 13 | MIMAT0000461 |
| | AUAUUGGC | | |
| hsa-miR-4777-3p* | AUACCUCAUCUAG | 14 | MIMAT0019935 |
| | AAUGCUGUA | | |
| hsa-miR-376c | AACAUAGAGGAAA | 15 | MIMAT0000720 |
| | UUCCACGU | | |
| hsa-miR-377-3p* | AUCACACAAAGGC | 16 | MIMAT0000730 |
| | AACUUUUGU | | |
| hsa-miR-3940-5p* | GUGGGUUGGGGCG | 17 | MIMAT0019229 |
| | GGCUCUG | | |
| hsa-miR-4649-5p* | UGGGCGAGGGGUG | 18 | MIMAT0019711 |
| | GGCUCUCAGAG | | |
| hsa-miR-494 | UGAAACAUACACG | 19 | MIMAT0002816 |
| | GGAAACCUC | | |
| hsa-miR-548ah-5p* | AAAAGUGAUUGCA | 20 | MIMAT0018972 |
| | GUGUUUG | | |
| hsa-miR-4778-5p* | AAUUCUGUAAAGG | 21 | MIMAT0019936 |
| | AAGAAGAGG | | |
| hsa-miR-575 | GAGCCAGUUGGAC | 22 | MIMAT0003240 |
| | AGGAGC | | |
| hsa-miR-425-3p | AUCGGGAAUGUCG | 23 | MIMAT0001343 |
| | UGUCCGCCC | | |
| hsa-miR-934* | UGUCUACUACUGG | 24 | MIMAT0004977 |
| | AGACACUGG | | |
| hsa-miR-22-5p | UGGAGUGUGACAA | 25 | MIMAT0000421 |
| | UGGUGUUUG | | |
| hsa-miR-30a-3p | CAGUGCAAUGUUA | 26 | MIMAT0000425 |
| | AAAGGGCAU | | |
| hsa-miR-1282* | UCGUUUGCCUUUU | 27 | MIMAT0005940 |
| | UCUGCUU | | |
| hsa-miR-523-3p | GAACGCGCUUCCC | 28 | MIMAT0002840 |
| | UAUAGAGGGU | | |
| hsa-miR-30c-1-3p* | CUGGGAGAGGGUU | 29 | MIMAT0004674 |
| | GUUUACUCC | | |
| hsa-miR-483-5p | AAGACGGGAGGAA | 30 | MIMAT0004761 |
| | AGAAGGGAG | | |
| hsa-miR-2115-5p* | AGCUUCCAUGACU | 31 | MIMAT0011158 |
| | CCUGAUGGA | | |
| hsa-miR-211-3p* | GCAGGGACAGCAA | 32 | MIMAT0022694 |
| | AGGGGUGC | | |
| hsa-miR-3187-5p* | CCUGGGCAGCGUG | 33 | MIMAT0019216 |
| | UGGCUGAAGG | | |
| hsa-miR-3194-5p* | GGCCAGCCACCAG | 34 | MIMAT0015078 |
| | GAGGGCUG | | |
| hsa-miR-518d-5p* | CUCUAGAGGGAAG | 35 | MIMAT0005456, |
| | CACUUUCUG | | MIMAT0005455, |
| | | | MIMAT0002845 |
| hsa-miR-3189-5p* | UGCCCCAUCUGUG | 36 | MIMAT0019217 |
| | CCCUGGGUAGGA | | |
| hsa-miR-574-5p | UGAGUGUGUGUGU | 37 | MIMAT0004795 |
| | GUGAGUGUGU | | |
| hsa-miR-125b-2-3p | UCACAAGUCAGGC | 38 | MIMAT0004603 |
| | UCUUGGGAC | | |
| hsa-miR-151a-5p | UCGAGGAGCUCAC | 39 | MIMAT0004697 |
| | AGUCUAGU | | |
| hsa-miR-151b* | UCGAGGAGCUCAC | 40 | MIMAT0010214 |
| | AGUCU | | |
| hsa-miR-130a-3p | CAGUGCAAUGUUA | 41 | MIMAT0000425 |
| | AAAGGGCAU | | |
| hsa-miR-5095* | UUACAGGCGUGAA | 42 | MIMAT0020600 |
| | CCACCGCG | | |
| hsa-miR-373-3p | GAAGUGCUUCGAU | 43 | MIMAT0000726 |
| | UUUGGGGUGU | | |
| hsa-miR-1268b* | CGGGCGUGGUGGU | 44 | MIMAT0018925 |
| | GGGGGUG | | |
| hsa-miR-939 | UGGGGAGCUGAGG | 45 | MIMAT0004982 |
| | CUCUGGGGGUG | | |

### DISCUSSION

These results provide the first examination of miRNAs present in urinary exosomes. Exosomes are membrane-derived vesicles that encapsulate cytoplasmic contents and thus provide a view of both membrane and cytoplasmic-based biomolecules, including miRNAs. Previous studies of circulating miRNAs excreted in urine have demonstrated association between presence of disease and changes in the concentration of urinary-selected miRNAs [11]. However, the interpretation that those miRNAs are biomarkers for kidney disease is complicated by the fact that urinary miRNA is composed of a combination of both filtered miRNA (derived from the blood) as well as kidney-derived miRNA. For our studies, we used urinary exosomes to exclude miRNA filtered from the blood because plasma exosomes are too large to pass through the glomerulus, allowing us to identify only miRNA from the kidney.

In the present disclosure, 24 of the 45 miRNAs that associated with salt sensitivity or inverse salt sensitivity have never been reported as secreted miRNAs. Some of the miRNAs that we identified as potential biomarkers for SS and ISS hypertension have been reported to regulate known pathways implicated in hypertension, including PPARγ [12], EGFR [13], TGFβ-1 [14] and PTEN/PI3K signaling [15]. Since changes in miRNA expression may occur early in disease pathogenesis, we postulate that urinary exosomal miRNAs may be useful biomarkers for individuals with aberrant sodium regulatory pathways [6]. In addition, each segment of the human nephron has distinct sodium regulatory functions. The identification of exosomal membrane markers in fractionated nephron-specific exosomes will shed light on the specific gene regulatory function of urinary exosomal miRNA.

The kidney nephron-derived exosomal miRNAs that were identified in our study may associate with kidney-specific metabolic activity, particularly renal sodium metabolism. Exosomal miRNAs that did not vary between conditions tested may serve as renal-specific controls for future longitudinal studies. Plasma-specific exosomal biomarkers may be used to exclude plasma contamination from occult glomerular diseases.

One of the best methods for determining salt sensitivity of blood pressure to date has been measuring the blood pressure response to a chronic change in oral salt intake. Urinary surrogate markers, including renal proximal tubule cells, exosomes, and miRNA, hold promise for cost-effective methods to screen for salt sensitivity and inverse salt sensitivity.

The salt sensitivity of patients with or without essential hypertension has not clinically documented, especially in salt-sensitive normotensive patients, who by definition have blood pressures less than 140/90 mmHg (systolic/diastolic) on random salt intake. Almost half of the US population may be salt-sensitive.

The salt sensitivity of blood pressure in hypertensive and normotensive patients is not included in published statistics on the prevalence, awareness, treatment, or response to treatment. Salt sensitivity, independent of blood pressure, is a risk factor for cardiovascular morbidity and mortality and other diseases, for example, asthma, gastric carcinoma, osteoporosis, and renal dysfunction.

Sodium intake and cardiovascular morbidity and mortality have a J-shaped curve relationship. A low-salt diet may not be beneficial to everyone as a low salt intake, as with a high salt intake, has also been associated with increased cardiovascular risk. Some individuals have a paradoxical increase in blood pressure on a low-salt diet (inverse salt sensitivity).

### CONCLUSION

Investigating renal cellular pathophysiology through the study of intracellular biomarkers has the potential of providing a deeper understanding of how individuals express unique patterns of salt sensitivity of blood pressure. Ultimately, personalized therapeutic approaches to controlling salt-related illnesses will result from these new diagnostic techniques.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

[1] A. Morimoto, T. Uzu, T. Fujii, M. Nishimura, S. Kuroda, S. Nakamura, T. Inenaga, G. Kimura, Sodium sensitivity and cardiovascular events in patients with essential hypertension, Lancet 350 (1997) 1734-1737.
[2] R. M. Carey, C. D. Schoeffel, J. J. Gildea, J. E. Jones, H. E. McGrath, L. N. Gordon, M. J. Park, R. S. Sobota, P. C. Underwood, J. Williams, B. Sun, B. Raby, J. Lasky-Su, P. N. Hopkins, G. K. Adler, S. M. Williams, P. A. Jose, R. A. Felder, Salt sensitivity of blood pressure is associated with polymorphisms in the sodium-bicarbonate cotransporter, Hypertension 60 (2012) 1359-1366.
[3] R. A. Felder, M. J. White, S. M. Williams, P. A. Jose, Diagnostic tools for hypertension and salt sensitivity testing, Curr Opin Nephrol Hypertens 22 (2013) 65-76.
[4] J. W. Dear, J. M. Street, M. A. Bailey, Urinary exosomes: A reservoir for biomarker discovery and potential mediators of intra-renal signaling, Proteomics (2012).
[5] H. Valadi, K. Ekstrom, A. Bossios, M. Sjostrand, J. J. Lee, J. O. Lotvall, Exosome-mediated transfer of mRNAs and microRNAs is a novel mechanism of genetic exchange between cells, Nat Cell Biol 9 (2007) 654-659.
[6] M. Ciesla, K. Skrzypek, M. Kozakowska, A. Loboda, A. Jozkowicz, J. Dulak, MicroRNAs as biomarkers of disease onset, Anal Bioanal Chem 401 (2011) 2051-2061.
[7] K. C. Miranda, D. T. Bond, M. McKee, J. Skog, T. G. P\check{a}unescu, N. Da Silva, D. Brown, L. M. Russo, Nucleic acids within urinary exosomes/microvesicles are potential biomarkers for renal disease, Kidney Int 78 (2010) 191-199.
[8] H. W. Khella, M. Bakhet, Z. Lichner, A. D. Romaschin, M. A. Jewett, G. M. Yousef, MicroRNAs in Kidney Disease: An Emerging Understanding, Am J Kidney Dis (2012).
[9] J. J. Gildea, D. T. Lahiff, R. E. Van Sciver, R. S. Weiss, N. Shah, H. E. McGrath, C. D. Schoeffel, P. A. Jose, R. M. Carey, R. A. Felder, A Linear Relationship Between the Ex-vivo Sodium Mediated Expression of Two Sodium Regulatory Pathways as a Surrogate Marker of Salt Sensitivity of Blood Pressure in Exfoliated Human Renal Proximal Tubule Cells: The Virtual Renal Biopsy, Clin Chim Acta (2013).
[10] P. A. Gonzales, H. Zhou, T. Pisitkun, N. S. Wang, R. A. Star, M. A. Knepper, P. S. Yuen, Isolation and purification of exosomes in urine, Methods in Molecular Biology 641 (2010) 89-99.
[11] J. M. Lorenzen, I. Volkmann, J. Fiedler, M. Schmidt, I. Scheffner, H. Haller, W. Gwinner, T. Thum, Urinary miR-210 as a mediator of acute T-cell mediated rejection in renal allograft recipients, Am J Transplant 11 (2011) 2221-2227.
[12] A. Jiang, S. Zhang, Z. Li, R. Liang, S. Ren, J. Li, Y. Pu, J. Yang, miR-615-3p promotes the phagocytic capacity of splenic macrophages by targeting ligand-dependent nuclear receptor corepressor in cirrhosis-related portal hypertension, Exp Biol Med (Maywood) 236 (2011) 672-680.
[13] A. L. Teixeira, M. Gomes, R. Medeiros, EGFR signaling pathway and related-miRNAs in age-related diseases: the example of miR-221 and miR-222, Front Genet 3 (2012) 286.
[14] T. Yang, Y. Liang, Q. Lin, J. Liu, F. Luo, X. Li, H. Zhou, S. Zhuang, H. Zhang, MiR-29 mediates TGFβ1-induced extracellular matrix synthesis through activation of PI3K-AKT pathway in human lung fibroblasts, J Cell Biochem (2012).
[15] Y. Li, L. Gao, X. Luo, L. Wang, X. Gao, W. Wang, J. Sun, L. Dou, J. Li, C. Xu, M. Zhou, M. Jiang, J. Zhou, M. A. Caligiuri, C. Nervi, C. D. Bloomfield, G. Marcucci, L. Yu, Epigenetic silencing of microRNA-193a contributes to leukemogenesis in t(8;21) acute myeloid leukemia by activating the PTEN/PI3K signal pathway, Blood 121 (2013) 499-509.
[16] Gildea et al., Clin Biochem., epub May 2, 2013, Urinary exosome miRNome analysis and its applications to salt sensitivity of blood pressure.

What is claimed is:

1. A method for determining whether a subject is salt-sensitive or inverse salt-sensitive by detecting and measuring miRNAs from purified urinary exosomes, said method comprising detecting and measuring in a sample from said subject at least one urinary exosomal miRNA from the group consisting of miRNAs having the sequence of SEQ ID NOs:1-45:
1) wherein when the level of at least one miRNA having SEQ ID NOs:1-11 measured in the sample from said subject is higher than the level of said at least one miRNA having SEQ ID NOs:1-11 from a salt-resistant (SR) subject, is an indication that said subject is salt-sensitive (SS);
2) wherein when the level of at least one miRNA having SEQ ID NOs:12-24 measured in the sample from said subject is lower than the level of said at least one miRNA having SEQ ID NOs:12-24 in a SR subject or a control or standard, is an indication that said subject is SS;
3) wherein when the level of at least one miRNA having SEQ ID NOs:10 and 25-37 measured in the sample from said subject is higher than the level of said at least one miRNA having SEQ ID NOs:10 and 25-37 in a SR subject, is an indication that said subject is inverse-salt sensitive (ISS); and
4) wherein when the level of at least one miRNA having SEQ ID NOs:7, 17, 18, and 38-45 measured in the sample from said subject is lower than the level of said at least one miRNA having SEQ ID NOs:7, 17, 18, and 38-45 in a SR subject or a control or standard, is an indication that said subject is ISS.

2. The method of claim 1, wherein each of said miRNAs having SEQ ID NOs:1-45 is detected and measured.

3. The method of claim 1, wherein parts 1) and 2) are combined.

4. The method of claim 1, wherein parts 3) and 4) are combined.

5. The method of claim 1, wherein parts 1), 2), 3), and 4) are combined.

6. The method of claim 5, wherein each of said miRNAs having SEQ ID NOs:1-45 is detected and measured.

7. The method of claim 1, part 1), wherein at least two of said miRNAs having SEQ ID NOs:1-11 is measured.

8. The method of claim 7, wherein each of said miRNAs having SEQ ID NOs:1-11 is measured.

9. The method of claim 7, wherein when at least two of said miRNAs having SEQ ID NOs:1-11 is higher is an indication said subject is SS.

10. The method of claim 9, wherein each of said miRNAs having SEQ ID NOs:1-11 is higher.

11. The method of claim 1, part 2), wherein at least two of said miRNAs having SEQ ID NOs:12-24 is measured.

12. The method of claim 11, wherein each of said miRNAs having SEQ ID NOs:12-24 is measured.

13. The method of claim 11, wherein when at least two of said miRNAs having SEQ ID NOs:12-24 is lower is an indication said subject is ISS.

14. The method of claim 13, wherein each of said miRNAs having SEQ ID NOs:12-24 is lower.

15. The method of claim 1, part 3), wherein at least two of said miRNAs having SEQ ID NOs:10 and 25-37 is measured.

16. The method of claim 15, wherein each of said miRNAs having SEQ ID NOs:10 and 25-37 is measured.

17. The method of claim 15, wherein when at least two of said miRNAs having SEQ ID NOs:10 and 25-37 is higher is an indication said subject is ISS.

18. The method of claim 15, wherein each of said miRNAs having SEQ ID NOs:10 and 25-37 is higher.

19. The method of claim 1, part 4), wherein at least two of said miRNAs having SEQ ID NOs:7, 17, 18, and 38-45 is measured.

20. The method of claim 19, wherein each of said miRNAs having SEQ ID NOs:7, 17, 18, and 38-45 is measured.

21. The method of claim 19, wherein when at least two of said miRNAs having SEQ ID NOs:10 and 25-37 is lower is an indication said subject is ISS.

22. The method of claim 21, wherein when each of said miRNAs having SEQ ID NOs:10 and 25-37 is lower is an indication said subject is ISS.

23. The method of claim 1, wherein when said miRNA having SEQ ID NO:7 is higher, said subject is SS.

24. The method of claim 1, wherein when said miRNA having SEQ ID NO:7 is lower, said subject is SS.

25. The method of claim 1, wherein SEQ ID NO:7 is detected and measured.

26. The method of claim 1, wherein SEQ ID NOs:10, 17, and 18 are detected and measured.

27. The method of claim 1, wherein when said subject is diagnosed to be SS or ISS, a treatment regimen is designed and implemented to treat said subject.

28. The method of claim 27, wherein said treatment regimen comprises administration of at least one drug selected from the group consisting of diuretics, combination diuretics, ACE inhibitors, angiotensin II receptor blockers, calcium channel blockers, central agonists, peripheral-acting adrenergic blockers, and direct renin inhibitors.

29. The method of claim 27, further wherein a dietary regimen is implemented to regulate salt intake and levels in said subject.

30. The method of claim 1, wherein when said subject is diagnosed as SS or ISS a dietary regimen is implemented to regulate salt intake and levels in said subject.

31. The method of claim 1, wherein said diagnosis is substantiated by measuring blood pressure response to a change in dietary salt intake or by measuring at least one surrogate marker selected from the group consisting of plasma renin activity (PRA), atrial natriuretic peptide (ANP), and brain natriuretic peptide (BNP).

32. A method of treating salt-sensitivity or inverse salt-sensitivity in a subject in need thereof, said method comprising: determining whether a subject is salt-sensitive or inverse salt-sensitive by detecting and measuring miRNAs from purified urinary exosomes, said method comprising detecting and measuring in a sample from said subject at least one urinary exosomal miRNA from the group consisting of miRNAs having the sequence of SEQ ID NOs:1-45:
1) wherein when the level of at least one miRNA having SEQ ID NOs:1-11 measured in the sample from said subject is higher than the level of said at least one miRNA having SEQ ID NOs:1-11 from a salt-resistant (SR) subject, is an indication that said subject is salt-sensitive (SS);
2) wherein when the level of at least one miRNA having SEQ ID NOs:12-24 measured in the sample from said subject is lower than the level of said at least one miRNA having SEQ ID NOs:12-24 in a SR subject or a control or standard, is an indication that said subject is SS;
3) wherein when the level of at least one miRNA having SEQ ID NOs:10 and 25-37 measured in the sample from said subject is higher than the level of said at least one miRNA having SEQ ID NOs:10 and 25-37 in a SR subject, is an indication that said subject is inverse-salt sensitive (ISS);
4) wherein when the level of at least one miRNA having SEQ ID NOs:7, 17, 18, and 38-45 measured in the sample from said subject is lower than the level of said at least one miRNA having SEQ ID NOs:7, 17, 18, and 38-45 in a SR subject or a control or standard, is an indication that said subject is ISS; and
5) administering a treatment regimen to the subject based on the level of said detected and measured miRNAs compared to the level of said miRNAs in an SR subject or a control or standard.

33. A method for detecting kidney-specific miRNAs in urinary exosomes prepared in the absence of a protease inhibitor, said method comprising:
1) centrifuging a urine sample that is less than about four hours old at about 17,000×g for 30 minutes to remove cells and debris;
2) obtaining a supernatant from step 1 and submitting said supernatant to ultracentrifugation at about 200,000×g for about 1 hour and optionally submitting a pellet obtained from said centrifugation to one or more rounds of ultracentrifugation;
3) resuspending the pellet of step 2 into phosphate buffered saline and isolating total RNA from said resuspended pellet; and
4) submitting said RNA to microarray analysis and detecting and quantifying miRNA in said sample.

34. A kit for determining whether a subject is salt-sensitive or inverse salt-sensitive, said kit comprising probes for at least one of an miRNA having SEQ ID NOs:1-45 and an instructional material for the use thereof.
